(19) United States
(12) Patent Application Publication
(10) Pub. No.: US 2008/0058762 A1
(43) Pub. Date: Mar. 6, 2008

(54) Induction And Maintenance Of Tolerance To Composite Tissue Allografts

(75) Inventor: Maria Z. Siemionow, Cleveland, OH (US)

Correspondence Address: MEDLEN & CARROLL, LLP, 101 HOWARD STREET, SUITE 350, SAN FRANCISCO, CA, 94105, US

(73) Assignee: THE CLEVELAND CLINIC FOUNDATION, Cleveland, OH (US)

(21) Appl. No.: 11/773,803

(22) Filed: Jul. 5, 2007

Related U.S. Application Data
(62) Division of application No. 10/427,013, filed on Apr. 30, 2003, now abandoned.
(60) Provisional application No. 60/382,680, filed on May 22, 2002.

(51) Int. Cl. A61M 31/00; A61K 39/395

(52) U.S. Cl. 604/522; 128/898; 424/144.1; 424/154.1

(57) ABSTRACT

The present invention provides a clinically-applicable approach for inducing long-term, donor-specific tolerance to donor antigens, especially in recipients of CTA and/or solid organ transplants, without the requirement for patient preconditioning, without the need for chronic immunosuppressive regimens, and without the occurrence of GVHD. In particular, a method is provided for inducing donor-specific tolerance in a semi-allogeneic or a fully-allogeneic transplant recipient by administering to the recipient a therapeutically effective amount of an immunosuppressive agent that depletes T cells and a therapeutically effective amount of anti-αβ T cell receptor antibodies, and implanting an allograft into the recipient.

This application claims the benefit of U.S. Provisional Application Ser. No. 60/382,680, filed May 22, 2002, the entire disclosure of which is hereby incorporated by reference.

### BACKGROUND OF THE INVENTION

Recent advances in the field of clinical organ transplantation have made procedures such as heart, kidney and liver transplantation widely available to patients with end-organ disease. Various immunosuppressive drug regimens designed to control acute and chronic graft rejection play a vital role in facilitating modern transplantation. However, the requirement for life-long immunosuppressive drug therapy to prevent chronic graft rejection carries a significant risk of infectious and neoplastic complications due to the nonspecific immunosuppressive effects of these drugs. The high morbidity and mortality associated with the chronic use of immunosuppressive agents has been a prohibiting factor in transplantation of composite tissue allografts (CTAs), such as hand, knee joint, larynx, and the like.

In contrast to solid organ allografts, CTAs are neurovascularized allografts of tissues that include structural, functional, and aesthetic units of integumentary and musculoskeletal elements. Although not vital to life, CTAs are important to those who deal with the functional restoration of musculoskeletal defects. CTAs are unique in that they are a heterogeneous histological milieu of tissue elements, with each component possessing different antigen expression and presentation mechanisms. For example, CTAs can be comprised of several tissue types including skin, subcutaneous tissue, nerve and vascular tissues, bone, muscle, fascia, cartilage, and the like. In addition, CTAs can contain immunocompetent elements, such as bone marrow and lymph nodes, that can hasten the graft rejection processes and/or result in graft versus host disease (GVHD). The heterogeneous nature of CTAs not only affects the immune reactivity of these allogeneic tissues, but also defines potential immunomodulating strategies that may be different from those currently used in solid organ transplantation. That the vast majority of attempts at CTA transplantation have been unsuccessful illustrates the difficult barrier associated with a neurovascularized allograft comprised of a variety of tissues.

Therefore, it is a goal of CTA transplantation to provide effective, non-toxic treatments that can ensure indefinite survival of all graft components, including skin, muscle, bone, vessels, nerve, tendon and bone marrow. In particular, there is a need for the reliable induction and maintenance in the recipient of immunological tolerance to donor-specific antigens without the need for chronic immunosuppressive regimens or patient preconditioning (such as by whole body irradiation or the like). Successful achievement of long-term immunological tolerance in the challenging CTA model could provide an important advance in the field of transplantation of both solid organ and CTA transplants, with significant health and economic benefits.

### SUMMARY OF THE INVENTION

The present invention provides a new approach for inducing long-term, donor-specific tolerance to donor antigens, especially in recipients of CTA transplants although not limited thereto, without the requirement for patient preconditioning, without the need for chronic immunosuppressive regimens, and without the occurrence of GVHD. The methods according to the invention are fully applicable to transplantation of any type of allograft, including solid organs such as, but not limited to, heart, lung, kidney, liver, pancreas, and the like, skin, hematopoietic tissue, lymphoid tissue, and the like, without limitation.

In particular, the embodiments of the methods according to the invention are fully clinically applicable to transplantation in human recipients and, for example, are adaptable to take into account such uncertainties as the timing of the availability of allograft transplants for human recipients, and the like. For example, in some embodiments, methods are provided in which a long-term tolerance-inducing and maintenance protocol can be initiated at about the time of transplantation to about 24 hours prior to transplantation, preferably at about the time of transplantation to about 12 hours prior to transplantation, more preferably from about 12 hours to about 24 hours prior to transplantation. In other embodiments, a protocol can be initiated at about an hour prior to transplantation to at about the time of transplantation. In other embodiments, a protocol can be initiated at about one day to about three days after transplantation.

Embodiments of the methods according to the invention are applicable to semi-allogeneic transplants such as, but not limited to, transplantation between related donor/recipients that are partially-mismatched at a major histocompatibility complex (MHC) class I or class II locus, and to fully-allogeneic transplants such as, but not limited to, transplantation between unrelated, fully mismatched MHC donor/recipient, including xenogeneic transplants to humans.

In one embodiment, a method is provided for inducing donor-specific tolerance in an allograft transplant recipient that comprises administering to the transplant recipient a therapeutically effective amount of an immunosuppressive agent that depletes T cells, preferably mature T cells, and also administering to the transplant recipient a therapeutically effective amount of anti-αβ T cell receptor antibodies. The immunosuppressive agent and the anti-αβ T cell receptor antibodies are administered in an amount, at a frequency, and for a duration of time sufficient to induce donor-specific tolerance in the recipient. The immunosuppressive agent is preferably an inhibitor of the calcineurin pathway of T cell activation such as, but not limited to, cyclosporine A (CsA), FK-506, and the like; or other inhibitors of IL-2 production such as, but not limited to, rapamycin and the like, and combinations of the foregoing. More preferably, the immunosuppressive agent is CsA.

In a preferred embodiment, the allograft transplant is a composite tissue allograft and, more preferably, the allograft includes donor bone, including the donor bone marrow. The immunosuppressive agent and the anti-αβ T cell receptor antibodies are administered for a period of time sufficient to induce donor-specific tolerance in the recipient. Donor-specific tolerance is evidenced by long-term to indefinite survival of the allograft after transplantation and treatment, and is observable by the induction and maintenance of hematopoietic mixed donor-recipient chimerism in the recipient. As used herein, mixed chimerism is used to described a state in which tissue or cells from a donor are able to live and function within a recipient host. The invention further provides an isolated hematopoietic cell from a recipient of a composite tissue allograft that includes donor bone and donor bone marrow, where the hematopoietic cell is a chimeric cell exhibiting characteristics of both the donor and the recipient.

In a preferred embodiment, the immunosuppressive agent and the anti-αβ T cell receptor antibodies are administered as a short course of therapy that can be initiated prior to transplantation, alternatively at transplantation, alternatively about one to about three days after transplantation and, preferably, continues for a short time period after transplantation. In an embodiment of the invention that is particularly useful for semi-allogeneic transplantation, the immunosuppressive agent and the anti-αβ T cell receptor antibodies are initially administered at about the time of transplantation to about 24 hours prior to transplantation, preferably about 12 hours to about 24 hours prior to transplantation. Administration of the immunosuppressive agent and the anti-αβ T cell receptor antibodies are then administered daily for about 100 days, about 50 days, about 35 days, about 21 days, about 14 days, preferably about 7 days or, especially, for about 5 days after transplantation.

In another embodiment of the invention that is particularly useful for fully-allogeneic transplantation, the immunosuppressive agent and the anti-αβ T cell receptor antibodies are initially administered at about one hour prior to transplantation to at about the time of transplantation. Administration of the immunosuppressive agent and the anti-αβ T cell receptor antibodies are then administered daily for about 100 days, about 50 days, about 35 days, about 21 days, about 14 days, preferably about 7 days or, especially, for about 5 days after transplantation. For fully allogeneic transplantation, it is more preferable to administer the immunosuppressive agent and the anti-αβ T cell receptor antibodies beginning at the time of transplantation, in order to avoid the occurrence of GVHD in these recipients.

In another embodiment, the immunosuppressive agent and the anti-αβ T cell receptor antibodies are initially administered from one to three days after transplantation, and daily administration continues for a period of time of about 100 days, about 50 days, about 35 days, about 21 days, about 14 days, preferably for about 7 days or, especially, for about 5 days after transplantation.

In alternative embodiments, the immunosuppressive agent and the anti-αβ T cell receptor antibodies can be administered independently on a daily and/or non-daily basis during the treatment period of time, depending on the type of transplant, the type of donor, the condition of the recipient, and other factors, according to the judgement of the practitioner as a routine practice, without departing from the scope of the invention.

The invention further provides a combination of pharmaceutical compositions for depletion of T cells in a recipient, comprising an effective amount of a pharmaceutical composition that comprises an immunosuppressive T cell-depleting agent, and an effective amount of a pharmaceutical composition that comprises anti-αβ TCR⁺ T cell receptor antibodies, wherein administration of the combination to the recipient results in elimination of about 50% to about 99.9%, preferably about 75% to about 95%, more preferably about 80% to about 90% of T cells circulating in peripheral blood of the recipient. Alternatively, the pharmaceutical composition can comprise an effective amount of both the immunosuppressive agent and the anti-αβ TCR⁺ T cell receptor antibodies.

Unexpectedly, it has been discovered that treating a CTA transplant recipient with a combination of the immunosuppressive agent and the anti-αβ T cell receptor antibodies results in induction of a long-term stable hematopoietic mixed donor-recipient chimerism in the recipient and long-term to indefinite survival of the allograft. By long term is meant a period of time greater than 100 days, preferably greater than 300 days, more preferably greater than 720 days and, most preferably, lifelong survival of the allograft after cessation of the treatment.

In an embodiment of the invention, the donor is a mammal of a first species and the recipient is a mammal of a second species. In a further embodiment, the donor and the recipient are of the same species. In yet a further embodiment, the recipient is a primate. In a preferred embodiment, the recipient is a human. In embodiment employing human recipients, the anti-αβ TCR receptor antibodies preferably comprise human antibodies to human αβ TCR⁺ T cell receptors. The anti-αβ TCR receptor antibodies are preferably monoclonal antibodies which can be humanized antibodies, but are preferably fully human polyclonal or monoclonal antibodies to human anti-αβ TCR receptors.

### BRIEF DESCRIPTION OF THE DRAWINGS

FIG. 1 illustrates the survival time of semi-allogeneic hindlimb transplants in positive (isograft) and negative (allograft) control groups and immunosuppressive treatment groups under a 35-day treatment protocol. Mean values indicate days following cessation of the treatment.

FIG. 2 illustrates the survival time of semi-allogeneic hindlimb transplants under different treatment intervals, indicating indefinite survival of semi-allogeneic allografts under the 35-day, the 14-day and the 7-day (protocol 7-I) CsA/αβ-TCR mAb treatment protocols.

FIG. 3 illustrates hematoxylin and eosin stained sections of skin from rejected CsA-only treated limb allograft (3A), skin from a non-rejected combined CsA/αβ-TCR mAb treated limb allograft (3B) and a rejection control limb allograft that received no treatment (3C).

FIG. 4 illustrates intravital microscopy images (×1800) from the control cremaster muscles (A), composite-tissue isograft (B) and allograft rejection controls (C), and composite-tissue allograft (CTA) under combined CsA/αβ-TCR mAb therapy (D) at day 7 post-transplantation. The upper images illustrate leukocyte-endothelial interactions (rolling, adherent, and transmigrated polymorphonuclear neutrophils in post-capillary venules (40 μm). External (arrowheads) and internal (small arrows) venular diameters are indicated. The ratio is the endothelial edema index (EET), indicating vascular occlusion. Below are the corresponding images after FITC albumin injection to reveal vessel permeability index (PI) (A1-D1). Control cremaster muscles with normal venular diameter, normal EEI and normal PI (A and A1).

FIG. 5 illustrates the results of the mixed lymphocyte reaction assay in long-term semi-allogeneic graft survivors of a 35-day combined CsA/αβ-TCR mAb treatment protocol at 400 days after transplantation.

FIG. 6 illustrates the results of the mixed lymphocyte reaction assay in long-term semi-allogeneic graft survivors of a 7-day (protocol 7-I) combined CsA/αβ-TCR mAb treatment protocol at 8 weeks after transplantation.

FIG. 7 illustrates a flow cytometry determination of in vivo immunodepletion of αβ TCR⁺ cells evaluated in the peripheral blood of the semi-allogeneic grafted animals treated with CsA alone or with the combined CsA/αβ-TCR mAb therapy in the 35-day protocol.

FIGS. 8A and 8B, respectively, illustrate lymphoid chimerism to both the donor and recipient CD4⁺ and CD8⁺ T-cell subpopulations in a representative nonmyeloablated non-conditioned recipient treated with the combined CsA/αβ-TCR mAb therapy in the 35-day protocol. The FC determination of the donor-specific chimerism revealed the presence of double positive CD4^{PE}/RT-1^{nFITC} (6.72%) and CD8^{PE}/RT-1^{nFITC} (1.2%).

FIG. 9 illustrates double-positive donor CD4^{PE}/RT-1^{nFITC} (3.4%) (left) and CD8^{PE}/RT-1^{nFITC} (12.8%) (right) T-cell subpopulations at 150 days post-transplant in the 35-day combined CsA/αβ-TCR mAb protocol.

FIG. 10 illustrates transplantation of the CTA across a major histocompatibility complex (MHC) barrier. (A) Before transplantation, donor LBN rat (left); recipient LEW rat (right). (B) After transplantation, LEW recipient of hindlimb allograft from LBN donor. (C) LEW recipient of LBN limb allograft at 650 days after transplantation, under the 35-day combined CsA/αβ-TCR mAb protocol, showing no signs of rejection and acceptance of the skin graft from the LBN donor. At the same time, third-party grafts were rejected.

FIG. 11 illustrates the limb allograft survival curve under 7 days (protocol 7-II) of the combined CsA/αβ-TCR mAb therapy in LEW recipients receiving hindlimb transplants across a strong MHC barrier, i.e., from fully mismatched BN donors.

FIG. 12 illustrates the results of the mixed lymphocyte reaction assay in long-term fully-allogeneic graft survivors of a 7-day (protocol 7-II) combined CsA/αβ-TCR mAb treatment protocol at 120 days after transplantation.

FIGS. 13A, 13B and 13C, respectively, illustrate flow cytometric triple-staining analysis of the donor-specific chimerism in long-term fully-allogeneic graft survivors of a 7-day (protocol 7-II) combined CsA/αβ-TCR mAb treatment protocol at 120 days after transplantation, showing that 1.3% of recipient peripheral blood mononuclear cells (PBMC) were CD8⁺ cells of donor origin; 7.6% of the recipient PBMC were CD4⁺ cells of donor origin; and 16.5% of the recipient PBMC were CD45RA⁺ B cells of donor origin, respectively.

FIG. 14 illustrates a flow cytometry determination of in vivo immunodepletion of αβ-TCR⁺ peripheral blood T cells under the 5-day (5-II), 7-day (7-II), and 21-day (21-II) Protocols using the combined CsA/αβ-TCR mAb therapy.

FIG. 15 illustrates peripheral blood lymphoid chimerism in fully-allogeneic recipients treated with the combined CsA/αβ-TCR mAb therapy in the (5-II), 7-day (7-II), and 21-day (21-II) protocols. The FC determination of the donor-specific chimerism revealed the presence of double positive CD4^{PE}/RT-1^{nFITC} (12.3%) and CD8^{PE}/RT-1^{nFITC} (9.6%) cells under the 5-II protocol, double positive CD4^{PE}/RT-1^{nFITC} (9.4%) and CD8^{PE}/RT-1^{nFITC} (8.7%) cells under the 7-II protocol, and double positive CD4^{PE}/RT-1^{nFITC} (10.1%) and CD8^{PE}/RT-1^{nFITC} (6.2%) cells under the 21-II protocol.

FIG. 16 illustrates the kinetics of the rise in the level of chimeric donor/recipient cells in the peripheral blood of the combined treatment allotransplant recipients leading to indefinite graft survival. Treated recipients of fully-allogeneic transplants exhibited 5.3% chimeric CD4⁺ cells by about day 14 after transplantation. This level rose to 7.2% by about day 35 after transplantation, and continued to rise to about 14.7% by about day 100 after transplantation.

### DETAILED DESCRIPTION OF THE INVENTION

The present invention provides methods for achieving long-term allograft survival without chronic immunosuppression and without the requirement for recipient conditioning, without the need for chronic immunosuppressive therapy, and without the occurrence of GVHD. The methods described and claimed herein are especially useful for human patients requiring transplantation of non-vital organs, including, but not limited to, those needing skin replacement after devastating burn injuries, cancer patients who need “customized” replacement of large parts of their bodies, immobilized rheumatoid patients requiring replacement of several joints, children born with congenital defects, and the like. Moreover, embodiments of the methods of the invention can be useful for solid organ transplants and allografts for treatment of inborn errors of metabolism, leukemias, immunodeficiency syndromes, GVHD relapse, and the like, without limitation.

Embodiments of the invention comprise the administration of a combination of anti-αβ T cell receptor antibodies and an immunosuppressive agent capable of depleting T cells, preferably mature T cells, prophylactically or therapeutically. By “prophylactic,” it is meant the protection, in whole or in part, against allograft rejection. By “therapeutic,” it is meant the amelioration of allograft rejection itself, and the protection, in whole or in part, against further allograft rejection. The antibodies and immunosuppressive drugs, as used herein, include all biochemical equivalents thereof (i.e., salts, precursors, the basic form, and the like).

An established model for composite tissue transplantation is transplantation of the rat hindlimb, optionally combined with the cremaster muscle flap for observation of microcirculation in the graft, if desired. After transplantation, the microcirculatory hemodynamics of rejecting muscle and the effects of surgical trauma can be monitored in the cremaster muscle under an intravital microscopy system. In this model, we have previously demonstrated that immunosuppression by agents such as cyclosporine A, prednisone, anti-ICAM-1 and anti-LFA-1, or recipient radiation, can significantly reduce graft permeability, endothelial cell damage, and trafficking of specific leukocyte subsets during the acute phase of allograft rejection, resulting in extended survival of CTA. Despite these promising results, and until the present invention, long-term rejection-free allograft survival was still dependent on the use of chronic immunosuppressive agents.

Although the rat hindlimb model is employed herein to demonstrate certain embodiments of the present invention, the invention is not limited thereto. In particular, the teachings and examples herein will enable the practitioner to routinely practice other embodiments of the methods useful for inducing and maintaining tolerance to donor allograft antigens. That is, the invention is intended to encompass, without limitation, embodiments where the donor of the allograft transplant is a mammal of a first species and the recipient is a mammal of a second species, and where the donor and the recipient are mammals of the same species. In one embodiment of the invention, the recipient is a primate. In a preferred embodiment, the recipient is a human. Embodiments of the invention further encompass inducing and maintaining tolerance to donor allograft antigens where the donor is a non-human mammal such as, but not limited to, a pig, other primates or the like, and the recipient is a human. In a more preferred embodiment, both the donor and the recipient are human.

The recipient can be fully mismatched to the donor at one or more loci that affect graft rejection, such as at an MHC class I or II locus or a minor antigen locus. Alternatively, the recipient can be mismatched to the donor at a first locus that affects graft rejection, and matched, or tolerant of a mismatch, at a second locus that affects graft rejection.

The present invention provides methods for achieving long-term CTA survival without chronic immunosuppression or patient conditioning by employing a new approach based on the pivotal role of T cells in the rejection of allografts.

T cell recognition of foreign major histocompatibility complex (MHC) antigens plays a crucial role in the initiation of allograft rejection. T lymphocytes are classified as αβ or γδ depending on the type of disulfide-linked heterodimeric glycoprotein T cell receptor (TCR) displayed. The T cells responsible for most immune responses, including allograft rejection, are the T cells hearing αβ T cell receptors (αβ TCR⁺ cells). In one embodiment of the present invention, anti-αβ TCR antibodies, preferably monoclonal (mAb) anti-αβ TCR antibodies, are employed to specifically eliminate αβ TCR⁺ cells to create a window of immunological incompetence in the transplant recipient. While not being bound by theory, it is believed that subsequent repopulation of the recipient thymus by αβ TCR⁺ cells, in the presence of donor alloantigens, results in the induction of hematopoietic mixed donor-recipient chimerism in the transplant recipient and the long-term immunological tolerance demonstrated herein.

In previous studies employing the rat hindlimb transplantation model, treatment with the anti-αβ TCR mAb alone reduced T cell numbers in a dose-dependent manner, with a significant reduction after one dose of antibody, but T cell depletion did not progress further with continued antibody therapy. While not being bound by theory, it is believed that some T cells in the blood, lymphatic vessels or lymphatic organs, such as the spleen, lymph nodes thymus, or the like, may have escaped initial exposure to the depleting antibody and repopulated rapidly to reject the graft.

To address this apparent “T cell escape phenomenon,” embodiments of the present invention employ an immunosuppressive agent, in addition to the anti-αβ TCR mAb, to prevent the rejection response by reducing allograft-responsive T cell proliferation and enhancing the effectiveness of the depletion protocol. An immunosuppressive agent, as used herein, is an agent such as a chemical agent or a drug that, when administered at an appropriate dosage over an appropriate time period, results in the depletion of T cells, preferably mature T cells. The immunosuppressive agent is preferably an inhibitor of the calcineurin pathway of T cell activation such as, but not limited to, cyclosporine A (CsA), FK-506, and the like; or other inhibitors of IL-2 production such as, but not limited to, rapamycin and the like. Calcineurin inhibitors prevent IL-2 gene transcription, thereby inhibiting T cell-mediated IL-2 production, a key cytokine for T cell expansion.

In one embodiment of the invention, the combined use of anti-αβ T cell antibodies and the immunosuppressive agent CsA with a 35-day protocol described below, successfully depleted αβ TCR⁺ cells in the transplant recipients by more than 97%, and created a therapeutic window of αβ TCR⁺ cell immunological silence between days 21 and 35 after transplantation under a 35-day treatment protocol. After discontinuation of the immunosuppressive regimen, T cell levels returned to 84% of the pre-transplant level at day 64, producing clinically observed tolerance of transplanted limbs with no further immunosuppressive therapy. The role of vascular thymus transplantation was also investigated and it was determined that donor thymus did not play a critical role in extending survival of limb allografts under this protocol (data not shown).

In another embodiment of the invention, the combined use of anti-αβ T cell antibodies and the immunosuppressive agent CsA daily for only 21 days, or only 7 days (protocol 7-I), or only 5 days (protocol 5-I) after transplantation successfully depleted αβ TCR⁺ cells in the transplant recipients by virtually 100% at day 7 post-transplantation. The level of depletion in each protocol group remained at greater than 95% to day 35 post-transplantation, creating a 28 day therapeutic window of αβ TCR⁺ cell immunological silence, even when the combined treatment was administered for only 5 to 7 days after transplantation. T cell levels gradually rose until by day 63, they had returned to 50-84% of the pre-transplant level and, by day 90 post-transplantation, had returned to 90-95% of the pre-transplant level, producing clinically observed tolerance of transplanted limbs with no further immunosuppressive therapy.

Although it is considered virtually not possible to eliminate all αβ TCR⁺ cells by the combined immunosuppressive and antibody therapy, it is preferable that the combined treatment be effective to eliminate about 50% to about 99.9%, preferably about 75% to about 95%, more preferably about 80% to at least about 90% of the αβ TCR⁺ cells during the short course of therapy. The embodiments of the method of the invention provide significant depletion of the recipient T-cell population at the end of the immunodepleting therapy, as well as allow repopulation of the recipient T cell repertoire once the treatment protocol is withdrawn.

The anti-αβ T cell receptor antibodies employed in embodiments of the methods according to the invention are preferably monoclonal (mAb) αβ T cell receptor antibodies, and are generally commercially available or can be produced by known methods without undue experimentation. Non-monoclonal anti-αβ T cell receptor antibodies with suitable specificity and an efficacy similar to monoclonal αβ T cell receptor antibodies, or whose epitope overlaps that of the monoclonal antibody, are also suitable. It is also known that hybridomas producing monoclonal antibodies may be subject to genetic mutation or other changes while still retaining the ability to produce monoclonal antibody of the same desired specificity. The embodiments of the invention methods therefore encompass mutants, other derivatives and descendants of the hybridomas producing anti-αβ TCR mAbs. It is also known that a monoclonal antibody can be subjected to the techniques of recombinant DNA technology to produce other derivative antibodies, humanized or chimeric molecules or antibody fragments that retain the specificity of the original monoclonal antibody. Such techniques may involve combining DNA encoding the immunoglobulin variable region, or the complementarity determining regions (CDRs) of the monoclonal antibody with DNA coding the constant regions, or the constant regions plus framework regions, of a different immunoglobulin, for example, to convert a mouse-derived monoclonal antibody into one having largely human immunoglobulin characteristics. (See, for example, EP 184187A and GB 2188638A.) The embodiments of the invention also encompass humanized monoclonal antibodies to the αβ TCR epitopes. In a most preferred embodiment, fully human antibodies to human αβ TCR epitopes are employed in human recipients. These human antibodies can be polyclonal with suitable specificity and efficacy and, preferably, are human monoclonal antibodies.

Without being bound by theory, it is believed that, since the vascularized bone marrow is an integral part of limb allografts, progenitor cells of donor bone marrow origin become engrafted in the recipient lymphoid organs during immunosuppressive therapy according to embodiments of the invention, resulting in induction of hematopoietic mixed donor-recipient chimerism in the recipient. Circulating chimeric cells can be identified in the peripheral blood and/or lymphoid organs of the recipients by staining with a monoclonal antibody specific for a donor peripheral blood mononuclear cell (PBMC) antigen. A low level of hematopoietic chimerism as a mechanism of tolerance induction is supported by other studies in which we found that rats rejected uniformly skin flap allografts devoid of bone marrow despite the CsA/αβ-TCR mAb treatment. In contrast, skin grafts transplanted simultaneously with bone marrow of donor origin were accepted (over 80 days) across an MHC barrier.

The level of chimerism present in the peripheral blood of recipients showing indefinite allograft tolerance is about 2% to about 3% at about 7 days post transplant. The level of chimerism then rises to about 3% to about 6% at about 21 days post-transplantation, to about 10% at about day 35, and to about 15% to about 20% or more by about day 63 post-transplantation. At this stage, a stable multilineage (CD4, CD8 and CD45RA) chimerism is achieved. The level of chimerism in lymphoid organs can be as high as about 60% or more or as low as about 25% or less.

Although in the rat hindlimb model, about 15% to about 20% chimeric cells were sufficient to achieve indefinite allograft tolerance, other protocols in other animals including human recipients may achieve a different peripheral blood level of chimeric cells that is sufficient to achieve indefinite allograft tolerance. Thus, the level of chimerism for maintaining long-term allograft tolerance can vary and can be about 5% or greater, without limitation, depending on the individual modality employed.

In the rat hindlimb transplantation model described in the examples below, a 35-day protocol of combined anti-αβ TCR antibodies and CsA therapy in the recipients of limb allografts induced tolerance across a major histocompatibility barrier without the need for chronic immunosuppression or recipient conditioning, and without inducing GVHD. Tolerance was confirmed with all animals (n=12) under the CsA/αβ TCR mAb treatment protocol and these accepted skin grafts from their CTA donors 100 days after transplantation. Third-party grafts were rejected, verifying the immunocompetence of the transplant recipient. All animals (n=12) remain healthy and no clinical signs of rejection of the allograft have developed for greater than 750 days after transplantation. Complete skin sensation returned by 6 weeks, and the animals were able to use the limbs for support.

In contrast, untreated recipients rejected allografts within 5 to 10 days after transplantation, and recipients treated with CsA alone rejected allografts within two weeks of cessation of the immunosuppressive therapy (42-50 days). Hematoxylin and eosin-stained sections of skin from rejected limb allograft controls, and CsA alone treated limb allografts, showed severe inflammation and ulceration extending through the full thickness of the skin and moderate infiltration of mononuclear cells in the dermis. No histopathological signs of rejection were observed in the CsA/αβ TCR mAb treated limb allograft group.

Flow cytometry (FC) analysis of peripheral blood samples at 650 days after transplantation showed the presence of circulating donor double-positive CD4⁺/RT-1ⁿ (6.7%) and CD8⁺/RT-1ⁿ (1.2%) lymphoid chimeric cells.

Other embodiments of the invention, described in the examples below, provided the same CsA/αβ-TCR mAb treatment protocol for 21 days, 14 days, 7 days (protocol 7-I), or 5 days (protocol 5-I) only, with the same successful induction and maintenance of long-term tolerance to semi-allogeneic transplantation.

In another embodiment, using a 21-day (protocol 21-II), 7-day (protocol 7-II) or 5-day protocol (protocol 5-II), in which the combined CsA/αβ TCR mAb treatment was initiated at the time of transplantation, it was demonstrated that tolerance was induced and maintained in fully-allogeneic transplants in MHC fully-mismatched recipients. With just this short course of combined therapy, the fully-allogeneic allografts show no evidence of rejection over a period of 350 days. Donor specific tolerance was confirmed by a mixed lymphocyte reaction (MLR). The hematopoietic tolerance inducing cells (TICs) circulating in the peripheral blood of the recipient were identified by flow cytometry using a three-color immunostaining technique developed in our laboratory. In addition to CD4 and CD8 donor/recipient chimeric cells, CD90⁺ cells were identified as the permissive cell population (16.5%) facilitating tolerance induction under these protocols.

Regardless of the immunosuppressive protocols employed, all of the recipients of the combined CsA/αβ-TCR mAb treatment were immunocompetent, as verified by the mixed lymphocyte reaction described in the examples below, in which the treated, allotransplanted recipients uniformly rejected third part grafts. Moreover, the immunocompetence of these recipients was verified biologically, as none of the animals over the entire range of protocols (including all recipients of semi-allogeneic and fully-allogeneic transplants and the combined treatment protocols) showed signs of disease, including viral infection or lymphoma formation, over 720 days post-transplant (which is a typical life span for a rat).

In a preferred embodiment the immunosuppressive T cell deleting agent and the anti-αβ TCR⁺ T cell receptor antibodies are administered to the transplant recipient as a combination of pharmaceutical compositions for depletion of T cells in the recipient, the combination comprising an effective amount of a pharmaceutical composition that comprises an immunosuppressive T cell-depleting agent, and an effective amount of a pharmaceutical composition that comprises anti-αβ TCR⁺ T cell receptor antibodies, wherein administration of the combination to the recipient results in elimination of about 50% to about 99.9%, preferably about 75% to about 95% and, more preferably about 80% to at least about 90% of T cells circulating in peripheral blood of the recipient. In another embodiment, the immunosuppressive T cell deleting agent and the anti-αβ TCR⁺ T cell receptor antibodies are administered as a pharmaceutical composition that comprises both the agent and the antibodies.

The immunosuppressive agent(s) and/or the antibodies useful in embodiments of the invention can be a pharmaceutically acceptable analogue or prodrug thereof, or a pharmaceutically acceptable salt of the immunosuppressive agent(s) or antibodies disclosed herein, which are effective in inducing long-term, donor specific tolerance to allografts. By prodrug is meant one that can be converted to an active agent in or around the site to be treated.

Treatment will depend, in part, upon the particular therapeutic composition used, the amount of the therapeutic composition administered, the route of administration, and the cause and extent, if any, of the disease.

The antibodies and immunosuppressive agent(s) described herein, as well as their biological equivalents or pharmaceutically acceptable salts can be independently or in combination administered by any suitable route, including oral, subcutaneous and parenteral administration. Examples of parenteral administration include intravenous, intraarterial, intramuscular, intraperitoneal, and the like. The manner in which the agents are administered is dependent, in part, upon whether the treatment is prophylactic or therapeutic. Although more than one route can be used to administer a particular therapeutic composition, a particular route can provide a more immediate and more effective reaction than another route. Accordingly, the described routes of administration are merely exemplary and are in no way limiting.

The dose of immunosuppressive agent and anti-αβ T cell receptor antibodies administered to an animal, particularly a human, in accordance with embodiments of the invention, should be sufficient to effect the desired response in the animal over a reasonable time frame. It is known that the dosage of therapeutic agents depends upon a variety of factors, including the strength of the particular therapeutic composition employed, the age, species, condition or disease state, and the body weight of the animal. Moreover, the dose and dosage regimen will depend mainly on whether the inhibitors are being administered for therapeutic or prophylactic purposes, separately or as a mixture, the type of biological damage to the host, the type of host, the history of the host, and the type of inhibitors or biological active agent. The size of the dose will be determined by the route, timing and frequency of administration as well as the existence, nature and extent of any adverse side effects that might accompany the administration of a particular therapeutic composition and the desired physiological effect. It is also known that various conditions or disease states, in particular, chronic conditions or disease states, may require prolonged treatment involving multiple administrations. Therefore, the amount of the agent and/or antibodies must be effective to achieve an enhanced therapeutic index. It is noted that humans are generally treated longer than mice and rats with a length proportional to the length of the disease process and drug effectiveness. The therapeutic purpose is achieved when the treated hosts exhibit improvement against disease or infection, including but not limited to improved survival rate of the graft and/or the host, more rapid recovery, or improvement in or elimination of symptoms. If multiple doses are employed, as preferred, the frequency of administration will depend, for example on the type of host and type of disease. The practitioner can ascertain upon routine experimentation which route of administration and frequency of administration are most effective in any particular case. Suitable doses and dosage regimens can be determined by conventionally known range-finding techniques. Generally, treatment is initiated with smaller dosages, which are less than the optimum dose of the compound. Thereafter, the dosage is increased by small increments until the optimum effect under the circumstances is reached.

In view of the teachings of the examples below for the rat hindlimb allograft transplant model, one of ordinary skill in the art will be guided to determining a suitable treatment regimen with routine experimentation. The immunosuppressive agent and the anti-αβ T cell receptor antibodies are administered for a period of time sufficient to induce donor-specific tolerance in the recipient. In a preferred embodiment, the immunosuppressive agent and the anti-αβ T cell receptor antibodies are administered as a short course of therapy that can begin prior to transplantation, alternatively at transplantation, alternatively about one to about three days after transplantation and, preferably, continues for a short time period after transplantation.

In a preferred embodiment, the immunosuppressive agent and the anti-αβ T cell receptor antibodies are administered as a short course of therapy that can be initiated prior to transplantation, alternatively at transplantation, alternatively about one to about three days after transplantation and, preferably, continues for a short time period after transplantation. In an embodiment of the invention that is particularly useful for semi-allogeneic transplantation, the immunosuppressive agent and the anti-αβ T cell receptor antibodies are initially administered about 12 to about 24 hours prior to transplantation. Administration of the immunosuppressive agent and the anti-αβ T cell receptor antibodies are then administered daily for about 100 days, about 50 days, about 35 days, about 21 days, about 14 days, preferably about 7 days or, especially, for about 5 days after transplantation.

In another embodiment of the invention that is particularly useful for fully-allogeneic transplantation, the immunosuppressive agent and the anti-αβ T cell receptor antibodies are initially administered during a period of time from about one hour prior to transplantation to at the time of transplantation. Administration of the immunosuppressive agent and the anti-αβ T cell receptor antibodies are then administered daily for about 100 days, about 50 days, about 35 days, about 21 days, about 14 days, preferably about 7 days or, especially, for about 5 days after transplantation. For fully allogeneic transplantation, it is more preferable to administer the immunosuppressive agent and the anti-αβ T cell receptor antibodies beginning at the time of transplantation, in order to avoid the occurrence of GVHD in these recipients.

In another embodiment, the immunosuppressive agent and the anti-αβ T cell receptor antibodies are first administered from one to three days after transplantation, and daily administration continues for a period of time of about 100 days, about 50 days, about 35 days, about 21 days, about 14 days, preferably for about 7 days or, especially, for about 5 days after transplantation.

In alternative embodiments, the immunosuppressive agent and the anti-αβ T cell receptor antibodies can be administered independently on a daily and/or non-daily basis during the treatment period of time, depending on the type of transplant, the type of donor, the condition of the recipient, and other factors, according to the judgement of the practitioner as a routine practice, without departing from the scope of the invention.

The immunosuppressive agent(s) and/or antibody compositions for use in embodiments of the invention generally include carriers. The carriers can be any of those conventionally used and are limited only by chemico-physical considerations, such as solubility and lack of reactivity with the compound, and by the route of administration. In addition to the following described pharmaceutical compositions, the therapeutic composition can be formulated as polymeric compositions, inclusion complexes, such as cyclodextrin inclusion complexes, liposomes, microspheres, microcapsules, and the like, without limitation.

The therapeutic composition can be formulated as a pharmaceutically acceptable acid addition salt such as, but not limited to, those derived from mineral acids such as, but not limited to, hydrochloric, hydrobromic, phosphoric, metaphosphoric, nitric and sulfuric acids, and the like, and organic acids such as, but not limited to, tartaric, acetic, citric, malic, lactic, fumaric, benzoic, glycolic, gluconic, succinic, and arylsulfonic, such as p-toluenesulfonic, and the like.

The pharmaceutically acceptable excipients described here, for example, vehicles, adjuvants, carriers or diluents, are well known and readily available. It is preferred that the pharmaceutically acceptable carrier be one which is chemically inert to the therapeutic composition and one that has no detrimental side effects or toxicity under the conditions of use.

The choice of excipient will be determined in part by the particular therapeutic composition, as well as by the particular method used to administer the composition. Accordingly, there is a wide variety of suitable formulations of the pharmaceutical composition used in the embodiments of the invention. For example, the non-limiting formulations can be injectable formulations such as, but not limited to, those for intravenous, subcutaneous, intramuscular, intraperitoneal injection, and the like, topical ointment formulations for application to the skin, including patches, corneal shields and ophthalmic ointments, and oral formulations such as, but not limited to, liquid solutions, including suspensions and emulsions, capsules, sachets, tablets, lozenges, and the like. Non-limiting formulations suitable for parenteral administration include aqueous and non-aqueous isotonic sterile injection solutions, including non-active ingredients such as anti-oxidants, buffers, bacteriostats, solubilizers, thickening agents, stabilizers, preservatives, surfactants, and the like. The solutions can include oils, fatty acids, including detergents and the like, as well as other well known and common ingredients in such compositions, without limitation.

Each of the foregoing suitable formulations contains an effective and/or predetermined amount of the active ingredient. Parenteral formulations can be presented in unit-dose of multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described, and the like.

The embodiments of the invention can include the co-administration of other pharmaceutically active compounds. By “co-administration” is meant administration before, concurrently with, such as in combination with the therapeutic composition in the same formulation or in separate formulations, or after administration of a therapeutic composition as described above. For example, corticosteroids such as, but not limited to, prednisone, methylprednisolone, dexamethasone, triamcinalone acetinide, and the like, or noncorticosteroid anti-inflammatory compounds such as, but not limited to, ibuprofen, flubiproben, or the like, can be co-administered. Similarly, vitamins, minerals and/or micronutrients can be co-administered.

The examples presented below described methods for induction of tolerance across the semi-allogeneic and fully-allogeneic MHC barriers in CTA transplants using a combined anti-αβ TCR antibodies and CsA therapy. The methods of induction of tolerance across fully mismatched MHC, as well as partially mismatched MHC, barriers has clinical relevance for human recipients of transplants from living or cadaver donors that can be animal or human. These protocols may be especially beneficial, as the demand for donor organs is growing rapidly and society would prefer procedures that do not doom the patient to a lifelong course of immunosuppressive medication. Introduction of short protocols of the embodiments according to the invention eliminate the need for chronic immunosuppression and would reduce the risk of debilitating side effects.

Based on findings presented herein, selectively deleting virtually the entire population of αβ-TCR⁺ cells in an adult animal created a window of immunological incompetence and reverted the immune system back to an immature developmental state. T cells repopulated in the presence of CTA donor antigens, and cells that reacted against them were deleted or rendered unresponsive. Indefinite allograft survival without chronic immunosuppression was achieved by selective elimination of one population of cells and subsequent T cell repopulation that perceived the transplanted limb as self. Without being bound by theory, it is believed that engraftment of the donor origin cells was facilitated by vascularized bone marrow. The advantage of these short-term treatment protocols over previous designs is that they may avoid the problems associated with pre-transplant recipient conditioning, post-transplant recipient irradiation, bone marrow transplantation and chronic immunosuppression, and graft-versus-host disease.

### EXAMPLES

To illustrate certain embodiments of the method of the invention, a rat hindlimb allotransplantation model was employed to demonstrate the induction and maintenance of long-term donor-specific tolerance across a semi-mismatched and a fully-mismatched MHC barrier. Exemplified embodiments employ the administration of a combination of monoclonal anti-αβ T cell receptor antibodies and cyclosporine A for depletion of T cells, under various treatment protocols. This model and the protocols described in the examples are directly clinically applicable to human transplantation.

The examples described herein are not intended to be limiting, as one skilled in the art would recognize from the teachings hereinabove and the following examples, that other immunosuppressive agents, other anti-αβ T cell receptor antibodies (including non-monoclonal antibodies to the αβ T cell receptor), other dosage and treatment schedules, and other animal and/or humans, all without limitation, can be employed, without departing from the scope of the invention as claimed.

The following animals, reagents, assays and techniques were employed in the examples.

Animals

Seven- to 10-week-old male Lewis-Brown-Norway (LBN, RT-1¹⁺ⁿ F1), Lewis (LEW, RT-1¹) and Brown-Norway (BN, RT-1ⁿ) rats were purchased from Harlan Sprague-Dawley (Indianapolis, Ind.). Equivalent ACI (AxC Irish, RT-1^{a}) animals were used as third party donor controls. Animals were housed in a barrier animal facility and cared for according to specific National Institutes of Health animal care guidelines.

Transplantation Technique

Transplantations of hindlimbs between donor and recipient were performed under pentobarbital (50 mg/kg intraperitoneal) anesthesia using a standard microsurgery procedure (Press, B. H. J. et al. 1986. Ann. Plast. Surg. 16, 313-321). Briefly, a circumferential skin incision was made in the proximal one third of the right hindlimb. The femoral artery and vein were dissected, clamped, and cut proximal to the superficial epigastric artery. The femoral nerve was dissected and cut 1 cm distal to the inguinal ligament. The biceps femoris muscle was transected to expose the sciatic nerve. The nerve was then cut proximal to its bifurcation.

The donor was prepared in a similar way. The right hindlimb was amputated at the midfemoral level. The donor limb was attached to the recipient limb by a 20-gauge intramedullary pin and a simple cerclage wire. All large muscle groups were sutured in juxtaposition. The iliac vessels of the donor and femoral vessels of the recipient were anastomosed under an operating microscope with 10-0 sutures by using a standard end-to-end microsurgical anastomosis technique. The femoral and sciatic nerves were repaired by using a conventional epineural technique with four 10-0 sutures.

Treatment Protocols

A. 35-Day Protocol

Thirty-three semi-allogeneic limb transplantations between LBN donor and LEW recipient were performed. Control isogeneic grafts (n=10) between LEW donor and LEW recipients, and fully allogeneic grafts (n=13) between ACI (RT-1^{a}) third party donor and LEW recipient, were also performed. The isogeneic and fully allogeneic graft recipients received no immunosuppressive treatment before or after transplantation. The semi-allogeneic (“allograft”) treatment regimens were a daily injection of cyclosporine A (CsA) (n=5) or a combination of CsA and an anti-αβ TCR monoclonal antibody (CsA/αβ-TCR mAb) (n=5). Cyclosporine A (Sandoz Pharmaceutics Inc., East Hanover, N.J.) was dissolved daily in PBS (Fisher Scientific, Pittsburgh, Pa.) to a concentration of 5 mg/ml and administered subcutaneously to recipient animals. The dosage of CsA for the allograft treatment regimens was 16 mg/kg CsA, administered 12 hours before transplantation and daily thereafter for the first week. The dosage of CsA was tapered to 8 mg/kg at the end of the first week and tapered weekly thereafter to 4 mg and 2 mg/kg, respectively. Intraperitoneal injection of mouse anti rat αβ TCR mAb (clone R73, Pharmingen, San Diego, Calif.) (250 μg) was administered 12 hours before transplantation, and daily thereafter for the first week. The dosage of anti-αβ TCR mAb was then tapered to 50 μg at the end of the first week and was given every 2 days during the second week and every 3 days during the last 3 weeks. The treatments continued to day 35 post-transplantation only.

B. 21-Day Protocol

Semi-allogeneic, fully allogeneic and isogeneic hindlimb transplants were performed, as described for the 35-day treatment protocol described above. The isogeneic and fully allogeneic graft recipients received no immunosuppressive treatment before or after transplantation. The semi-allogeneic treatment regimens were a daily injection of CsA or a combination CsA/αβ-TCR mAb. A subcutaneous dosage of 16 mg/kg CsA was administered 12 hours before transplantation and daily thereafter for the first week post-transplantation. The dosage of CsA was reduced to 8 mg/kg at the end of the first week and to 4 mg at the end of the second week. Intraperitoneal injection of anti-αβ TCR mAb (250 μg) was administered 12 hours before transplantation, and daily thereafter for the first week post-transplantation. The dosage of anti-αβ TCR mAb was then tapered to 50 μg at the end of the first week and was given every 2 days during the second week and every 3 days during the last week. The treatments were continued to day 21 post-transplantation only.

In an alternative protocol (21-II), the initial injections of the CsA and anti-αβ TCR⁺ TCR mAb were performed at the time of transplantation.

C. 14-Day Protocol

Semi-allogeneic, fully allogeneic and isogeneic hindlimb transplants were performed, as described for the 35-day treatment protocol described above. The isogeneic and fully allogeneic graft recipients received no immunosuppressive treatment before or after transplantation. The semi-allogeneic treatment regimens were a daily injection of CsA or a combination CsA/αβ-TCR mAb. A subcutaneous dosage of 16 mg/kg CsA was administered 12 hours before transplantation and daily thereafter for the first week post-transplantation. The dosage of CsA was reduced to 8 mg/kg at the end of the first week. Intraperitoneal injection of anti-αβ TCR mAb (clone R73) (250 μg) was administered 12 hours before transplantation, and daily thereafter for the first week post-transplantation. The dosage of anti-αβ TCR mAb was then tapered to 50 μg at the end of the first week and was given every 2 days during the second week. The treatments were continued to day 14 post-transplantation only.

7-Day Protocol (7-I)

Semi-allogeneic, fully allogeneic and isogeneic hindlimb transplants were performed, as described for the 35-day treatment protocol described above. The isogeneic and fully allogeneic graft recipients received no immunosuppressive treatment before or after transplantation. The semi-allogeneic treatment regimens were a daily injection of CsA or a combination CsA/αβ-TCR mAb. A subcutaneous dosage of 16 mg/kg CsA was administered 12 hours before transplantation and daily thereafter for 7 days post-transplantation. Intraperitoneal injection of anti-αβ TCR mAb (250 μg) was administered 12 hours before transplantation, and daily thereafter for 7 days post-transplantation. The treatments were continued to day 7 post-transplantation only.

7-Day Protocol (7-II)

This 7-day protocol was employed for fully-allogeneic hindlimb transplants between Brown Norway (BN) donor rats and Lewis (LEW) recipient rats, including the isogeneic controls, described for the 7-day (7-I) protocol above. The protocol differs from the 7-day (7-I) protocol described above only in the time of the initial introduction of the CsA and/or anti-αβ TCR mAb. In this protocol, the initial administration of the combined CsA/αβ-TCR mAb therapy was performed on the day of transplantation, at the beginning of the procedure or during the transplantation procedure during clamp release. Subsequent treatment with the combined CsA/αβ-TCR mAb therapy continued for 7 days after transplantation as in the 7-I protocol described above. This 7-II protocol was employed for fully-allogeneic transplantation. No GVHD was observed in any of the rats receiving the combined therapy. It was found previously that a small number of fully-allogeneic transplant recipients under the 7-I protocol exhibited GVHD.

5-Day Protocol (5-I)

Semi-allogeneic, fully allogeneic and isogeneic hindlimb transplants were performed, as described for the 35-day treatment protocol described above. The isogeneic and fully allogeneic graft recipients received no immunosuppressive treatment before or after transplantation. The semi-allogeneic treatment regimens were a daily injection of CsA or a combination CsA/αβ-TCR mAb. A subcutaneous dosage of 16 mg/kg CsA was administered 12 hours before transplantation and daily thereafter for 5 days post-transplantation. Intraperitoneal injection of anti-αβ TCR mAb (250 μg) was administered 12 hours before transplantation, and daily thereafter for 5 days post-transplantation. The treatments were continued to day 5 post-transplantation only.

5-Day Protocol (5-II)

This 5-day protocol was employed for fully-allogeneic hindlimb transplants between Brown Norway (BN) donor rats and Lewis (LEW) recipient rats, including the isogeneic controls, described for the 5-day (5-I) protocol above. The protocol differs from the 5-day (5-I) protocol described above only in the time of the initial introduction of the CsA and/or anti-αβ TCR mAb. In this protocol, the initial administration of the combined CsA/αβ-TCR mAb therapy was performed on the day of transplantation, at the beginning of the procedure or during the transplantation procedure during clamp release. Subsequent treatment with the combined CsA/αβ-TCR mAb therapy continued as for the 5-I protocol described above.

Postoperative Care and Evaluation of Functional Recovery

Daily body weight, skin temperature, hair growth and limb circumference measurements were taken. Erythema, edema, loss of hair, epidermolysis, and progressive limb shrinkage were accepted as physical signs of rejection. Function of the accepted limbs was evaluated on a weekly basis for the first 3 months and once a month thereafter by pin-prick test for sensor recovery (0=no sensation, 1=withdrawal above the knee, 2=withdrawal response between the knee and ankle, 3=withdrawal distal to the ankle), and by toe spread test (0=no movement, 1=any signs of movement, 2 abduction, 3=abduction and extension of the toes) and standard walking, track analysis for motor recovery.

Evaluation of the Microcirculatory Hemodynamics

The cremasteric muscle, an optional component of the hindlimb transplant, served as the “microcirculatory window” to monitor directly in vivo leukocyte trafficking between the vascular space and graft interstitium. The effect of surgical trauma, graft acceptance and rejection was measured as a shift in leukocyte population between rolling, sticking and transmigrating leukocytes and lymphocytes. Using a standard intravital microscopy system with a final magnification of 1800× the microcirculatory measurements of the following hemodynamic parameters were recorded: i) vessel diameters (the first, second and third-order arterioles, and first-order and postcapillary venules) were measured with a video image measurement device; ii) red blood cell (RBC) velocity of the major vessels was recorded using an optical Doppler flow velocimeter (Texas A&M University, Galveston, Tex.); iii) functional capillary densities were measured in the proximal, middle, and distal flap regions in nine visual fields at each preselected postcapillary venule site for a total of 27 fields per cremaster muscle flap.

Leukocyte-Endothelial Interactions

The images of rolling, adherent, and transmigrated leukocytes were counted and recorded for 2 minutes on the video tapes allowing for repeated evaluation and slow motion recordings for analysis of leukocyte trafficking and distribution. Endothelial Edema Index (ELI) was calculated as the ratio of the external to the internal vessel diameter and indicated vascular occlusion due to endothelial cell swelling, deposits of adherent leukocytes within the venular lumen, vessel wall damage, and microvascular injury.

Evaluation of Microvascular Permeability Index (PI)

A Zeiss 20T fluorescent microscope (Carl Zeiss, Goettingen, Germany) equipped with an incident-light illumination system (460-490 nm blue light band excitation filter from a mercury arc lamp) and closed-circuit video recording system consisting of an MTI silicon-intensifying target camera (MTI SIT-68), a Panasonic AG-1290 video recorder, and 19-inch Sony Trinitron television was used. Following injection of 0.2 ml/100 g body weight of FITC albumin, fluorescent images were digitized using a Kontron Elektronik KS 300 V2.00 image analysis system (Kontron Elektronik GmbH, Eching, Germany). Video images from the microscope camera were digitized at a 512 by 512 pixel resolution for each video frame. Average intensity of the pixels was calculated for the interstitium (10 to 50 μm from the venular wall) and the venular lumen. The permeability index (PI), indicating vascular albumin leakage, was the ratio of interstitial intensity to venular intensity.

Skin Grafting

For in vivo testing of the donor-specific tolerance and immunocompetence, skin grafts were taken from the recipient (LEW), donor (LBN) and third-party (ACI) rats and placed on the long-term survivors 100 days after treatment cessation. The standard skin-grafting procedure described by Billington, R. and R. Medawar (Trans. Bull. 4, 67, 1957) was used. The grafts were evaluated on a daily basis after transplantation for signs of rejection. Rejection was defined as the destruction of more than 80% of the graft.

Donor Specific Tolerance In Vitro: Mixed Lymphocyte Reaction (MLR) Assay

Tolerant long-term limb allograft survivors of 7 to 35 days of the combined CsA/αβ-TCR mAb treatment protocol were tested for in vitro responsiveness at 400 days post-transplantation by an MLR assay. Responder cells were freshly isolated from the peripheral blood of naive animals (LEW) and limb recipients (LEW) by gradient centrifugation with Histoplaque (Sigma, St. Louis, Mo.), harvested, washed using phosphate buffered saline (PBS), and resuspended in RPMI complete medium (Gibco, Gaithersburg, Md.) supplemented with 10% fetal calf serum (Gibco), 2 mM 1-glutamine, 5×10⁻⁵ M 2-beta mercaptoethanol, 10 mM HEPES, and penicillin (100 U/ml) and streptomycin (100 U/ml) (Gibco). 2×10⁵ cells were delivered in triplicate to the wells of 96-well round bottom tissue culture plates. Stimulator cells (0.5×10⁶ and 0.25×10⁶ cells) were isolated from spleens of the naive syngeneic (LEW), semi-allogeneic (LBN) and third party (ACI) rats by passing/mincing method. The cells were inactivated by mitomycin C (Sigma) for 30 min at 37° C. and 5% CO₂ in the air, followed by washing in complete medium, re-suspended in complete medium, and incubated for 72 hours with the responder cells at 37° C. and 5% CO₂ in the air. After 72 hours, cultures were pulsed with 1 μCi [³H] thymidine and incubated 12-18 hours at 37° C. and 5% CO₂ in the air. The cells were then harvested onto fiber filter mats and assessed by a beta counter. The Stimulation Index (SI) was determined by assessment of the ratio of the cpm generated in response to a given stimulator over the baseline cpm generated in response to the host.

Flow Cytometry

Flow cytometry (FC) analysis was performed according to the manufacturer's protocol (Becton Dickinson, San Diego, Calif.) with minor modifications. The peripheral blood samples of transplant recipients were collected into heparinized tubes on post-transplantation days 0, 7, 21, 35, 63, and at the time of initial signs of clinical rejection. The whole blood was incubated for 20-30 min in the dark at room temperature, with 5 μL of mouse anti-rat FITC-conjugated mAbs against mature T lymphocytes (Pharmingen), CD3 (clone G4.18); helper-inducer T cells, CD4 (clone OX-35); cytotoxic-suppressor T lymphocytes, CD8a (clone OX-8); natural killer lymphocytes, NKR-P1 (clone 3.2.3); and αβ TCR on T lymphocytes (clone R73) (Becton-Dickinson). After incubation, samples were incubated with FACS Lysing solution (Becton-Dickinson), centrifuged at 1500 rpm for 5 min, and fixed with 2% paraformaldehyde solution. For the assessment of lymphoid chimerism, combinations of CD4-phycoerythrin (PE) with mouse anti-rat RT-1ⁿ-FITC (clone MCA156, Sertec, UK) and CD8-PE with RT-1ⁿ-FITC antibodies were used. For RT-1ⁿ fluorescent staining, samples were pre-incubated for 5 min with purified anti-rat CD-32 (FcγII Block Receptor) antibody (1:20), then incubated with 5 μL of RT-1ⁿ for 30 min at 4 C, washed twice, stained with goat anti-mouse IgG-FITC conjugated antibody (rat adsorbed, Serotec) and incubated with mouse anti-rat CD4-PE or CD8-PE conjugated monoclonal antibody and processed as indicated above.

Statistical Analysis

The results, including the level of αβ-TCR⁺ T-cell depletion, MLR, SI and microcirculatory parameters of allograft rejection, were averaged for the groups on each evaluation day and assessed for significant differences using the two-tailed Student's t-test. Differences were considered statistically significant at p<0.05.

### Example 1

### Survival of Hindlimb Allografts Under Combined CsA/αβ-TCR mAb Treatment Protocols

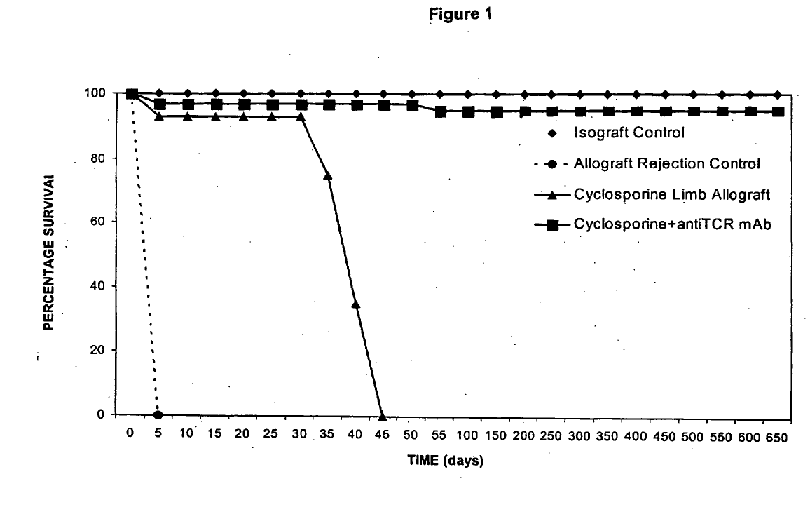

FIG. 1 illustrates the transplant survival time in the semi-allogeneic allograft and isograft controls and in experimental groups after immunosuppressive treatment under the 35-day treatment protocol. Isograft controls survived indefinitely. Allograft rejection controls rejected limbs at between days 5 and 7 post-transplantation. In the CsA-alone group, limb rejection occurred between 7 and 14 days after cessation of the 35-day treatment. Under combined CsA/αβ-TCR mAb treatment for 35 days, all limb allografts survived over 650 days, and have continued to survive over 720 days, without signs of rejection.

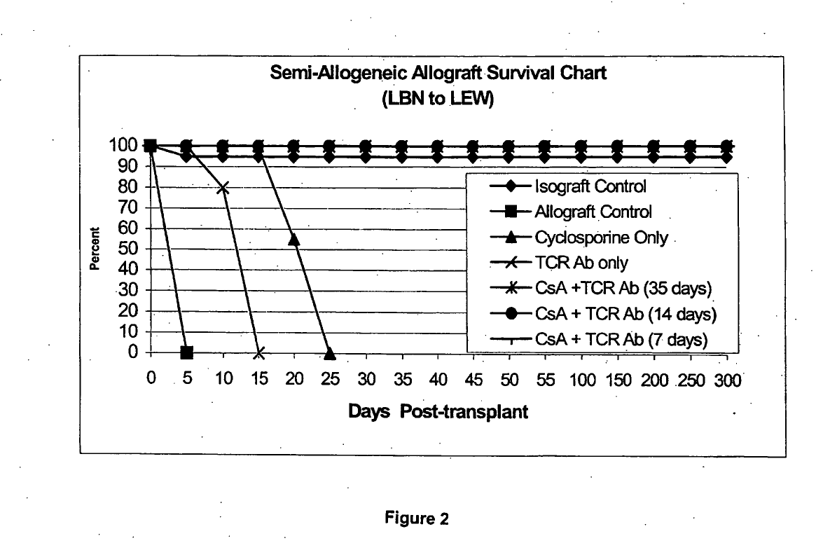

FIG. 2 illustrates the limb allograft survival curve under different treatment protocols, indicating indefinite survival (>300 days) of the semi-allogeneic allografts under the CsA/αβ-TCR mAb treatment protocol for 35 days, 14 days and 7-days only.

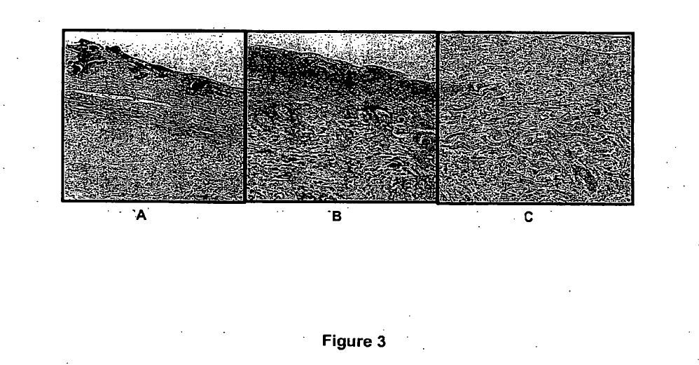

FIG. 3 illustrates hematoxylin and eosin stained sections of skin from rejected CsA-only treated limb allograft (3A), skin from a non-rejected combined CsA/αβ-TCR mAb treated limb allograft (3B) and a rejection control limb allograft that received no treatment (3C). Severe inflammation and ulceration extending through the full thickness of the skin are shown in A. Skin with mild inflammation affecting the dermis is shown in B. The inflammation is primarily made up of lymphocytes. The epidermis is fully intact. C shows necrosis of the skin extending into the hypodermis. The inflammation around the necrosis is primarily made up of neutrophils. The dermis has increased fibrosis.

### Example 2

### Microcirculatory Hemodynamics and Microvascular Permeability Index of Hindlimb Allografts Under Combined CsA/αβ-TCR mAb 35-Day Treatment are Comparable to Isograft Controls

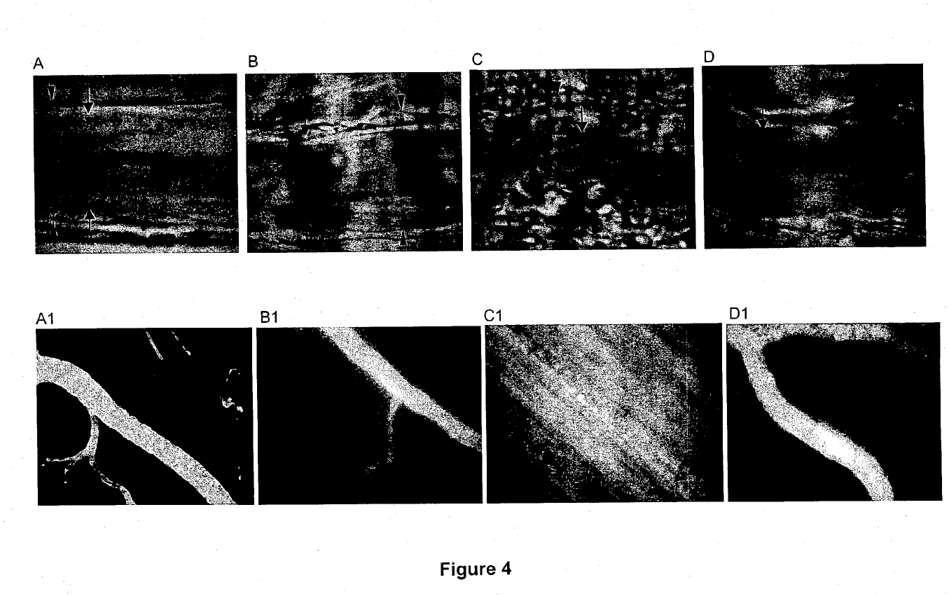

FIG. 4 illustrates intravital microscopy images (×1800) of microvessels in control and transplanted muscles at day 7 post-transplantation. Top images (FIG. 4, A-D) show leukocyte-endothelial interactions—rolling, adherent, and transmigrated polymorphonuclear neutrophils (PMNs) in posteapillary venules (40 μm) Arrowheads show the external, and small arrows the internal venular diameter. The ratio is the endothelial edema index (EEI), indicating vascular occlusion. Below are corresponding images after FITC albumin injection to reveal vessel permeability index (PI) (FIG. 4, A1-D1). Control cremaster muscles with normal venular diameter, normal EEI, and normal PI (FIG. 4, A and A1). FIG. 4, B and B1 illustrate the isograft control, with a 30% increase in rolling PMNs, a 60% increase in adherent PMNs, a 10% elevation in EEI, and a 12% increase in PI. FIG. 4, C and C1 illustrates the allograft rejection controls, with a 5-fold increase in rolling, a 10-fold increase in adherent PMNs, and a 9-fold increase in transmigrated PMNs, a 75% increase in venular EEI, and an 80% increase in PI. FIG. 4, D and D1 illustrate the allografts under combined CsA/αβ-TCR mAb 35-day treatment protocol at day 7 post-transplantation, having results comparable to the isograft controls, i.e., a 25% increase in rolling PMNs, a 40% increase in adherent PMNs, a 12% increase in transmigrating PMNs, an 8% increase in EEI, and a 10% increase in PI.

### Example 3

### Assessment of Functional Return

At 6 weeks post-transplantation, the pin-prick test revealed grade 3, full return of sensation, in all allograft recipients under combined CsA/αβ-TCR mAb 35-day treatment protocol. The animals had a normal sensation up to 720 days post-transplantation and use of their limbs as support. No toe-spread return was seen (grade 0), so walking-track analysis was inconclusive because of toe contracture.

### Example 4

### In Vitro Reactivity: MLR Assay, 35-Day Treatment Protocol

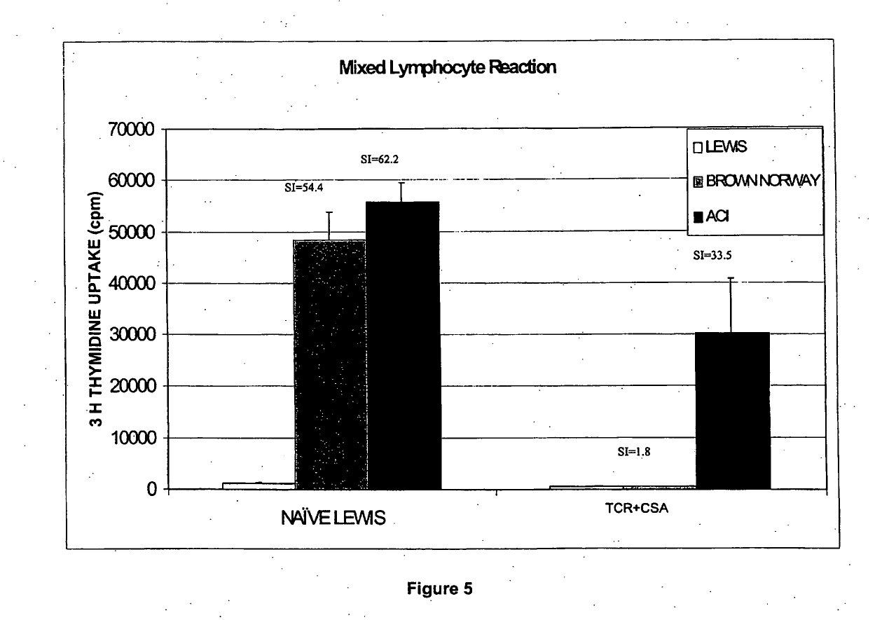

Donor specific tolerance in vitro was assessed in long-term survivors of the 35-day combined CsA/αβ-TCR mAb protocol at 400 days after hindlimb allotransplantation by the MLR assay directed against donor (LBN) and third-party antigens (ACI). Lymphocytes isolated from peripheral blood of recipients that accepted the allograft were not reactive to host (LEW) antigens (p<0.001) and donor (LBN) antigens (p<0.01) expressed on stimulator splenocytes, but revealed strong reactivity to splenocytes expressing third-party (ACI) alloantigens (p<0.05), as illustrated in FIG. 5.

### Example 5

### In Vitro Reactivity: MLR Assay, 7-Day Treatment Protocol (7-I), Semi-Allogeneic Transplant

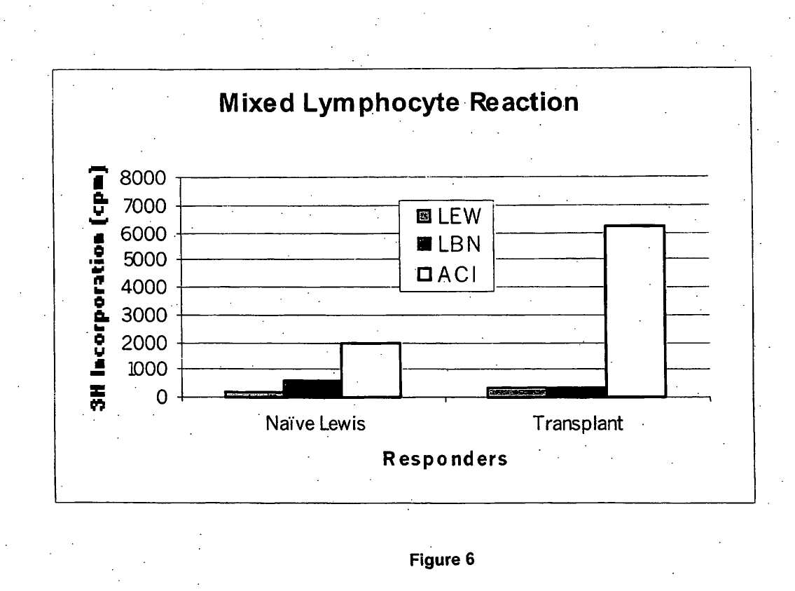

Donor specific tolerance in vitro was assessed in long-term semi-allogeneic graft survivors of the 7-day combined CsA/αβ-TCR mAb protocol (7-I) at 8 weeks after hindlimb transplantation by the MLR assay directed against donor (LBN) and third-patty antigens (ACT). Lymphocytes isolated from peripheral blood of (LEW) recipients that accepted the allograft showed a suppressed response against donor (LBN) antigens (p<0.05), and increased reactivity against third party antigens (p<0.05), as illustrated in FIG. 6.

### Example 6

### Immunosuppression of αβ-TCR⁺ Cells in Allograft Recipients Under the Combined CsA/αβ-TCR mAb 35-Day Treatment Protocol

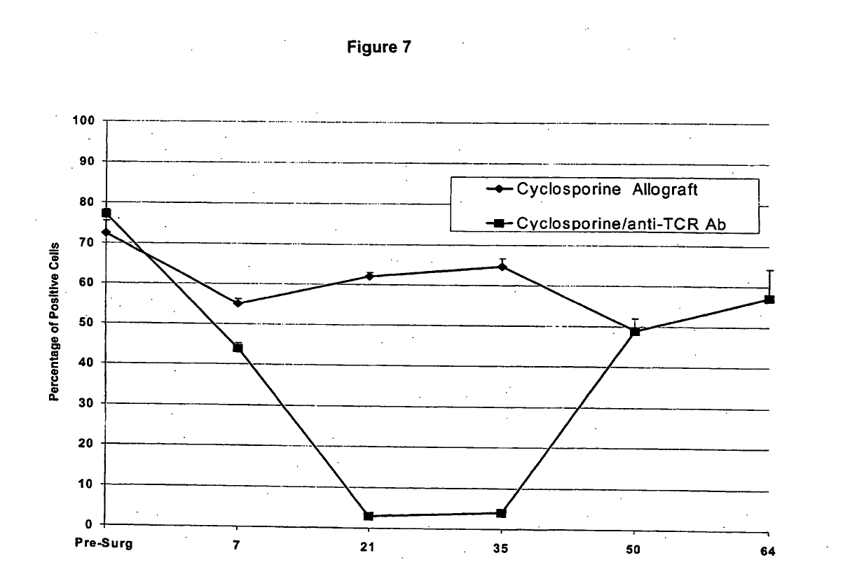

FIG. 7 illustrates the flow cytometry determination of the efficacy of the immunomodulating therapy of combined CsA/αβ-TCR mAb therapy in the peripheral blood of allograft recipients treated with CsA alone or with the combined CsA/αβ-TCR mAb therapy. The recipients of the combined therapy showed greater than 90% reduction of the αβ-TCR⁺ cells after 21 and 35 days of the combined therapy (p<0.05). Levels were observed to increase, by 16 and 19 fold, by day 50 and 64, respectively, due to re-population of new αβ-TCR⁺ cells. Treatment with CsA alone resulted in a reduction in αβ-TCR⁺ cells of only one-fold observed on day 35 and 1.5-fold on day 50.

### Example 7

### Donor-Specific Chimerism Demonstrated in Allograft Recipients Under the Combined CsA/αβ-TCR mAb 35-Day Treatment Protocol

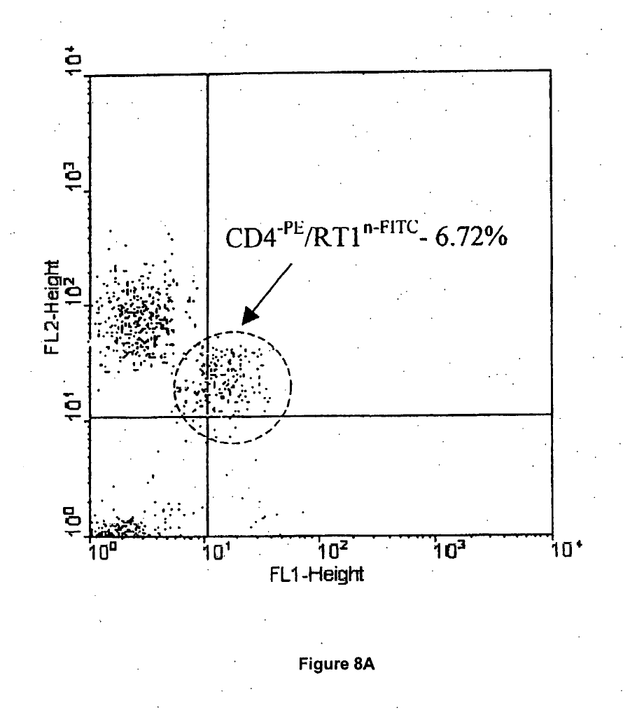

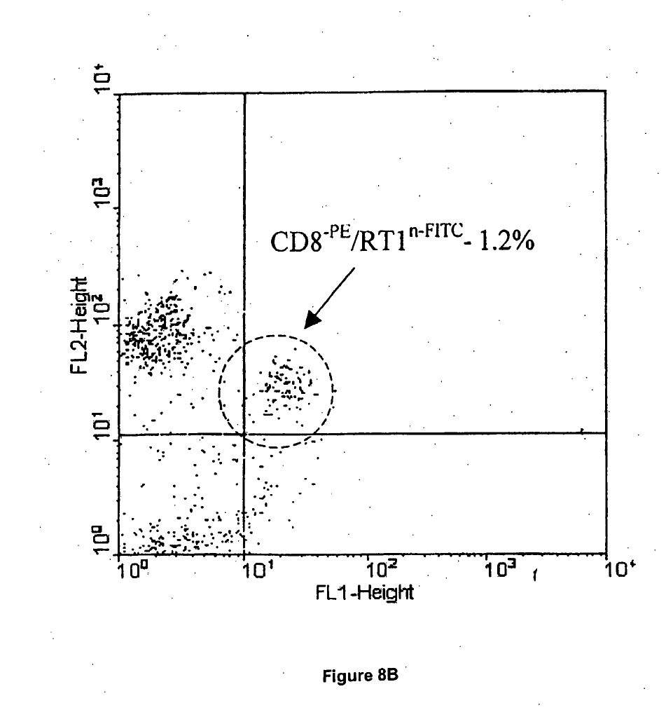

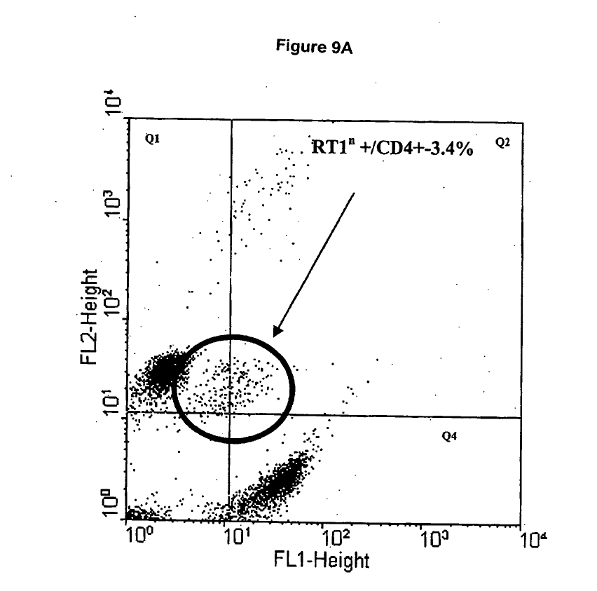

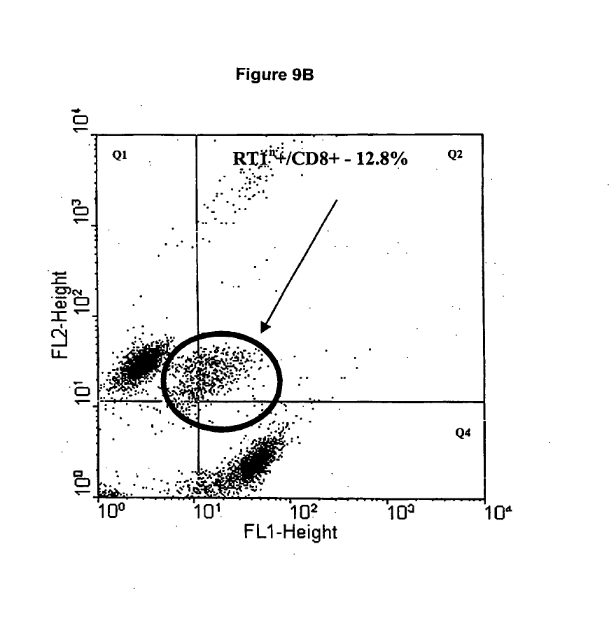

Flow cytometric analysis of the donor-specific chimerism revealed double-positive CD4^{PE}/RT-1^{nFITC} (6.7%) and CD8^{PE}/RT-1^{nFITC} (1.2%) T-cell subpopulations in the peripheral blood of hindlimb allograft recipients at 400 days post-transplantation (FIG. 8, A and B, respectively). As illustrated in FIG. 9, double-positive donor CD4^{PE}/RT-1^{nFITC} (3.4%) (left) and CD8^{PE}/RT-1^{nFITC} (12.8%) (right) T-cell subpopulations were present. In the isograft, allograft, and CsA-alone control groups, no chimeric cells were found in the periphery.

### Example 8

### In Vivo Evidence of Donor-Specific Tolerance and Immunocompetence: Skin Grafting

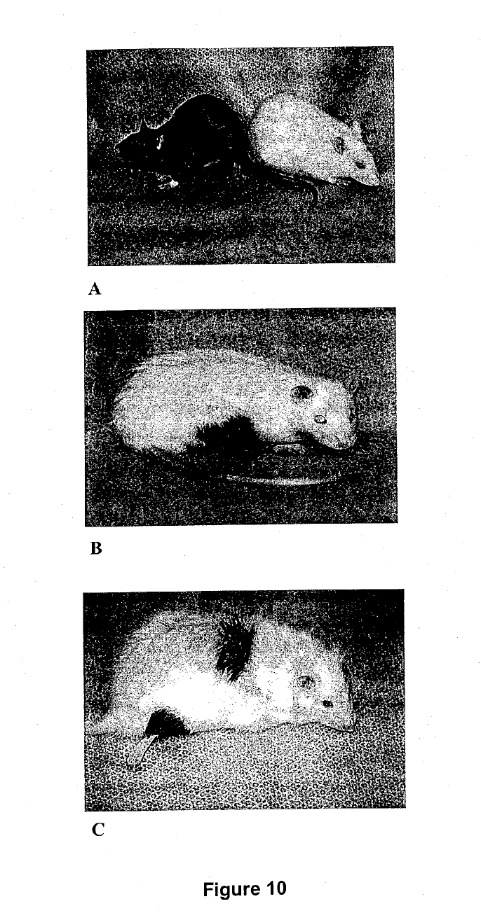

FIG. 10 illustrates semi-allogeneic transplantation of the CTA across a major histocompatibility complex (MHC) barrier. Donor-specific tolerance and immunocompetence was confirmed with all semiallogeneic grafted rats (n=5) under the 35-day combined CsA/αβ-TCR mAb therapy accepted secondary skin grafts from the donor (LBN) and rejected the third-party (ACI) alloantigens. A illustrates donor LBN rat (left); recipient LEW rat (right) before transplantation. B illustrates an after transplantation LEW recipient of hindlimb allograft from LBN donor. C illustrates a LEW recipient of an LBN limb allograft at 650 days after transplantation showing no signs of rejection and acceptance of the skin graft from the LBN donor. At the same time, third-party grafts were rejected.

### Example 9

### Survival of Fully Mismatched MHC Allografts, 7-Day (7-II) Protocol

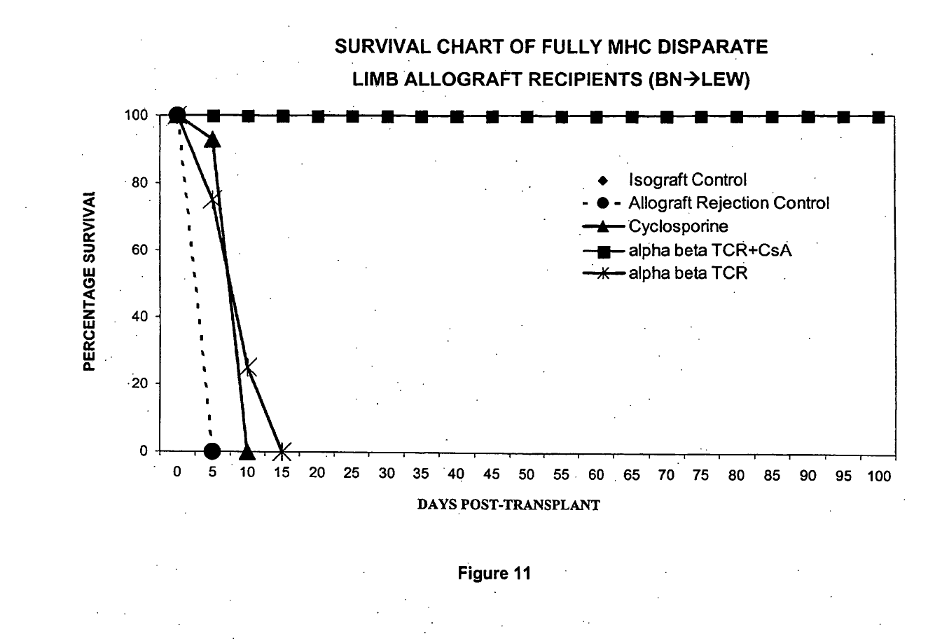

FIG. 11 illustrates the limb allograft survival curve under 7 days of the combined CsA/αβ-TCR mAb therapy (protocol 7-II) in LEW recipients receiving hindlimb transplants across a strong MHC barrier, i.e., from fully mismatched BN donors. The hindlimb grafts of the group of recipients receiving the combined CsA/αβ-TCR mAb showed indefinite survival (>300 days, still alive). In contrast, the fully allogeneic graft with no treatment was rejected in under 7 days post-transplantation; the CsA treatment only group rejected the grafts within 18-20 days post-transplantation; and the anti-αβ TCR mAb treatment only group rejected the grafts within 12-15 days post-transplantation.

### Example 10

### In Vitro Reactivity: MLR Assay, 7-Day Treatment Protocol, Fully-Allogeneic Transplant

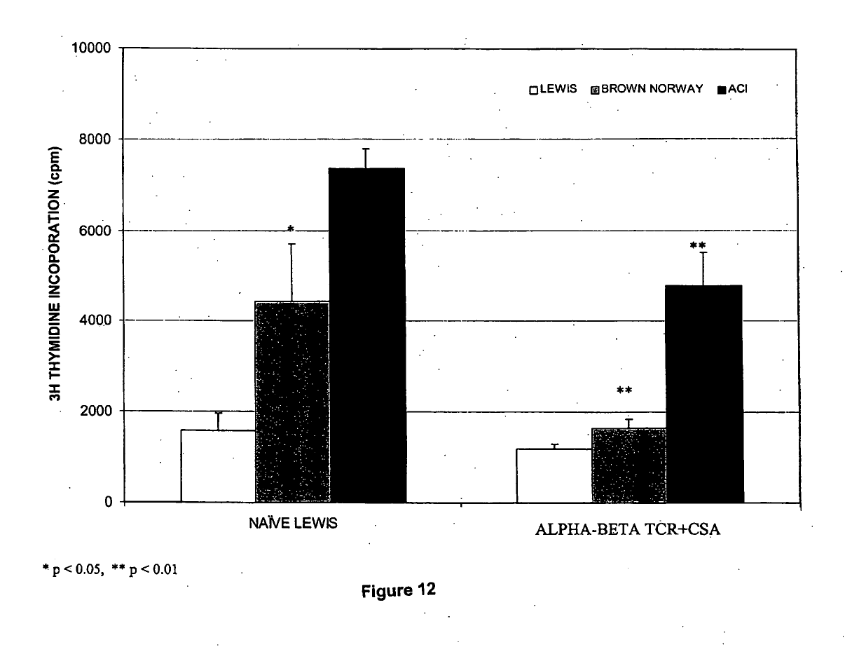

Donor specific tolerance in vitro was assessed in long-term fully-allogeneic graft survivors of the 7-day combined CsA/αβ-TCR mAb protocol (7-II) at 120 weeks after hindlimb transplantation by the MLR assay directed against donor (BN) and third-party antigens (ACI). Lymphocytes isolated from peripheral blood of (LEW) recipients that accepted the fully allogeneic grafts showed a suppressed response against donor (BN) antigens (p<0.01), and increased reactivity against third party antigens (p<0.01), as illustrated in FIG. 12.

### Example 11

### Donor-Specific Chimerism Demonstrated in Fully Allogeneic Graft Recipients Under the Combined CsA/αβ-TCR mAb 7-Day (7-II) Treatment Protocol

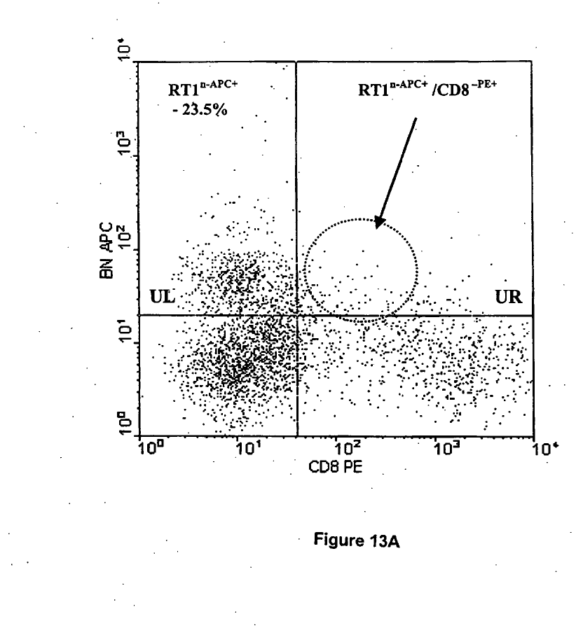

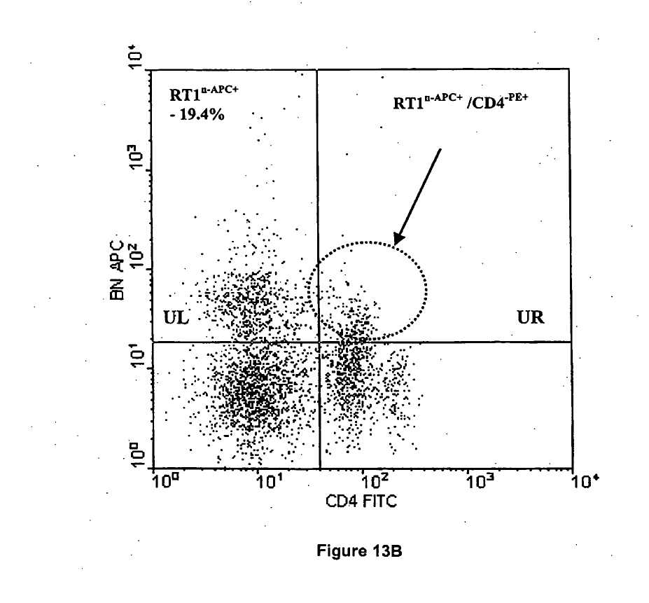

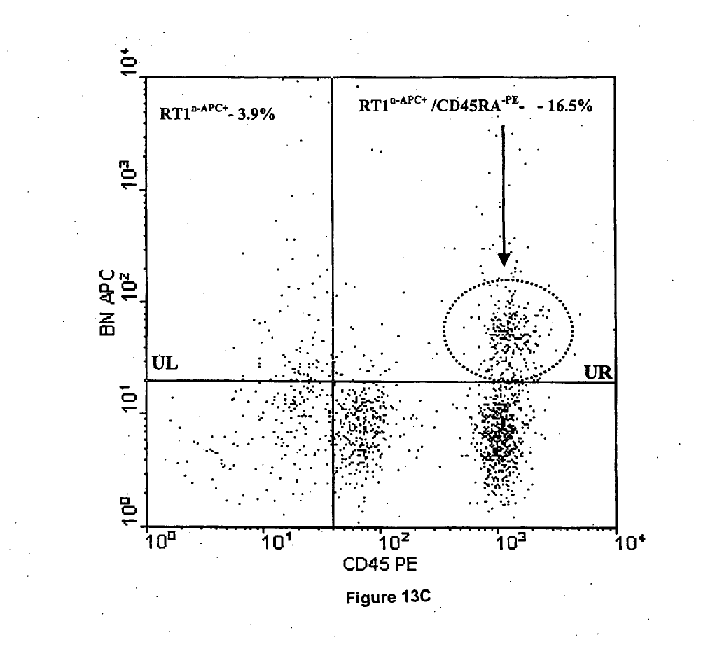

Flow cytometric analysis of the donor-specific chimerism using the triple staining technique revealed that, at 120 days post-transplantation, 1.3% of isolated recipient peripheral blood mononuclear cells (PBMC) were CD8⁺ cells of donor origin (FIG. 13, A); 7.6% of the isolated recipient PBMC were CD4⁺ cells of donor origin (B); and 16.5% of the recipient PBMC were CD45RA⁺ B cells of donor origin (C).

### Example 12

### Depletion of αβ-TCR⁺ Cells Under the 5-Day, 7-Day, and 21-Day Protocols

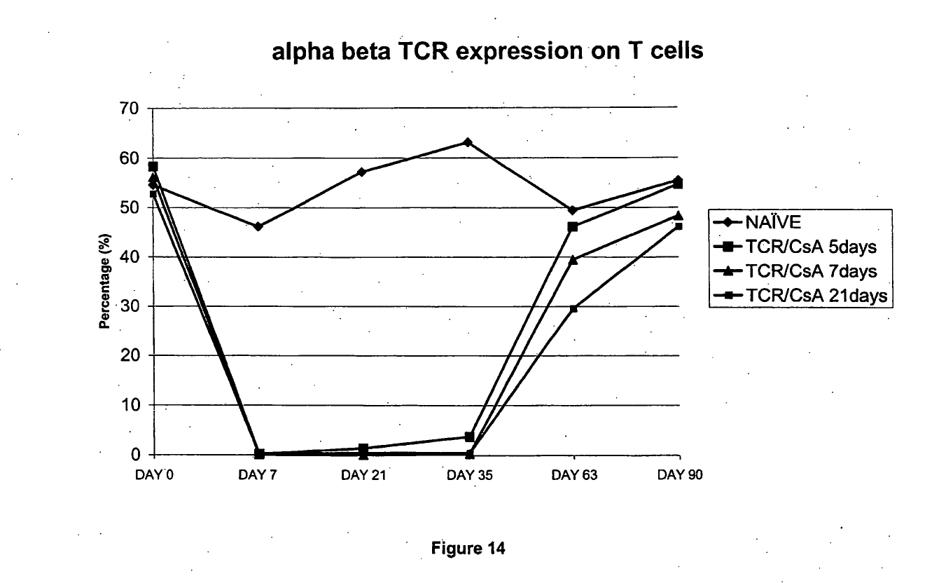

As illustrated in FIG. 14, the combined use of anti-αβ T cell antibodies and the immunosuppressive agent CsA daily for only 21 days (therapy begun at time of transplantation), or only 7 days (protocol 7-II), or only 5 days (protocol 5-II) after transplantation successfully depleted αβ TCR⁺ cells in the transplant recipients by virtually 100% at day 7 post-transplantation. The level of depletion in each protocol group remained at greater than 95% to day 35 post-transplantation, creating a 28 day therapeutic window of αβ TCR⁺ cell immunological silence, even when the combined treatment was administered for only 5 to 7 days after transplantation. T cell levels gradually rose until by day 63, they had returned to 50-84% of the pre-transplant level and, by day 90 post-transplantation, had returned to 90-95% of the pre-transplant level, producing clinically observed tolerance of transplanted limbs with no further immunosuppressive therapy.

### Example 13

### Donor-Specific Chimerism Demonstrated in Fully-Allogeneic Graft Recipients Under the Combined CsA/αβ-TCR mAb 5-Day (5-II), 7-Day (7-II) and 21-Day (“21-II”) Treatment Protocols

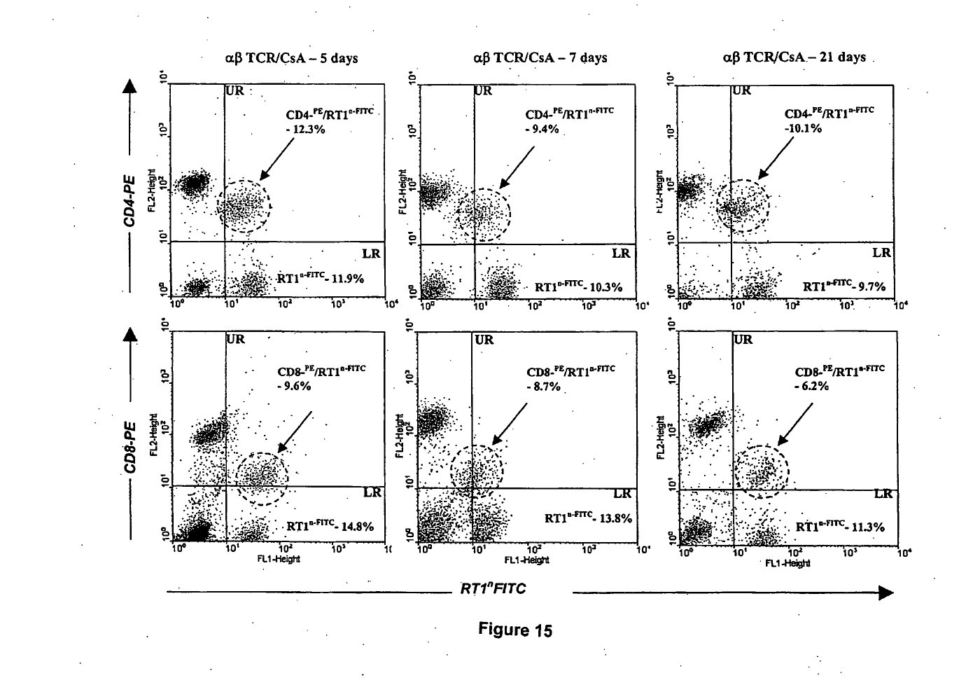

Flow cytometric analysis of the donor-specific chimerism using the triple staining technique, as illustrated in FIG. 15, revealed that at 120 days post-transplantation, using the 5-day (5-II) protocol, 9.6% of isolated recipient peripheral blood mononuclear cells (PBMC) were CD8⁺ cells of donor origin; 12.3% of the recipient PBMC were CD4⁺ cells of donor origin; and 14.8% of the recipient PBMC were B cells of donor origin. Similarly, using the 7-day (7-II) protocol, 8.7% of isolated recipient PBMC were CD8⁺ cells of donor origin; 9.4% of the recipient PBMC were CD4⁺ cells of donor origin; and 13.8% of the recipient PBMC were B cells of donor origin. Similarly, using a 21-day protocol in which the combined therapy was begun at the time of transplantation (“21-II”), 6.2% of isolated recipient PBMC were CD8⁺ cells of donor origin; 10.1% of the recipient PBMC were CD4⁺ cells of donor origin; and 11.3% of the recipient PBMC were B cells of donor origin.

### Example 14

### The Level of Chimeric Donor/Recipient Cells in the Peripheral Blood of Allotransplant Recipients Rises Over Time After Transplantation

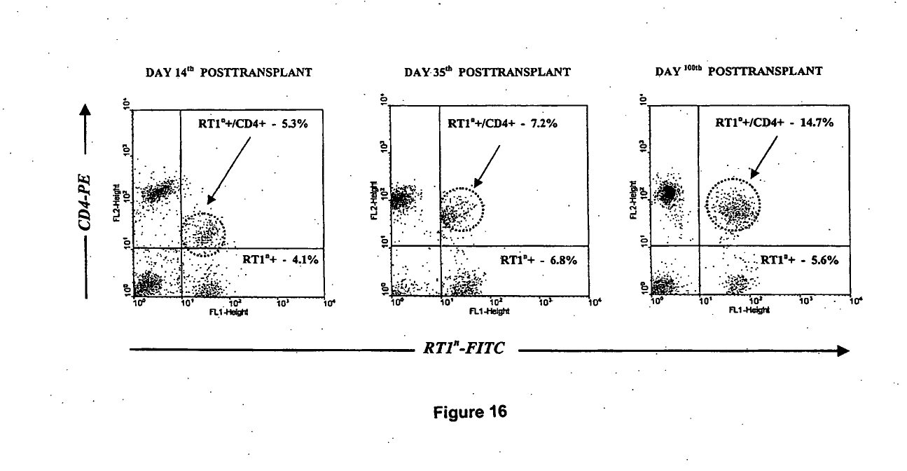

FIG. 16 illustrates the kinetics of the rise in the level of chimeric donor/recipient cells in the peripheral blood of the combined treatment allotransplant recipients leading to indefinite graft survival. The Figure illustrates that treated recipients of fully-allogeneic transplants exhibited 5.3% chimeric CD4⁺ cells by about day 14 after transplantation. This level rose to 7.2% by about day 35 after transplantation, and continued to rise to about 14.7% by about day 100 after transplantation. In this rat model, it is evident that a level of chimerism of about 15% by day 100 after transplant ensures indefinite transplant survival.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

1. A method for inducing donor-specific tolerance in an allograft transplant recipient, comprising:
(a) administering to a transplant recipient a therapeutically effective amount of an immunosuppressive agent that depletes T cells;
(b) administering to the transplant recipient a therapeutically effective amount of anti-αβ T cell receptor antibodies; and
(c) implanting an allograft into the transplant recipient.

2. The method of claim 1, wherein the graft is selected from the group consisting of a semi-allogeneic graft, a fully-allogeneic graft, and combinations thereof.

3. The method claim 1, wherein the anti-αβ TCR receptor antibodies are human antibodies to human αβ TCR⁺ T cell receptors.

4. The method of claim 1, wherein the anti-αβ TCR receptor antibodies are monoclonal antibodies.

5. The method of claim 4, wherein the anti-αβ TCR receptor antibodies are humanized monoclonal antibodies to human αβ TCR⁺ T cell receptors.

6. The method of claim 4, wherein the anti-αβ TCR receptor antibodies are human monoclonal antibodies to human αβ TCR⁺ T cell receptors.

7. The method of claim 1, wherein the allograft comprises a composite tissue allograft.

8. The method of claim 7, wherein the composite tissue allograft includes donor bone including donor bone marrow.

9. The method of claim 1, wherein the allograft comprises a solid organ allograft.

10. The method of claim 1, wherein the immunosuppressive agent depletes mature T cells.

11. The method of claim 10, wherein the immunosuppressive agent comprises an IL-2 production inhibitor.

12. The method of claim 10, wherein the immunosuppressive agent comprises a calcineurin inhibitor

13. The method of claim 1, wherein the immunosuppressive agent is selected from the group consisting of cyclosporine A, FK-506, rapamycin, and combinations thereof.

14. The method of claim 1, wherein the immunosuppressive agent and the anti-αβ T cell receptor antibodies are administered in an amount, at a frequency, and for a duration of time sufficient to induce donor-specific tolerance in the recipient.

15. The method of claim 1, wherein the immunosuppressive agent and the anti-αβ T cell receptor antibodies are both administered daily during the period of time.

16. The method of claim 1, wherein the immunosuppressive agent and the anti-αβ T cell receptor antibodies are administered independently on a daily or a non-daily basis during the period of time.

17. The method of claim 1, wherein the immunosuppressive agent and the anti-αβ T cell receptor antibodies are administered initially at about the time of transplantation to about 24 hours prior to transplantation.

18. The method of claim 17, wherein the immunosuppressive agent and the anti-αβ T cell receptor antibodies are initially administered at a time selected from the group consisting of about 12 hours to about 24 hours prior to transplantation, about at the time of transplantation to about 12 hours prior to transplantation, about at the time of transplantation to about one hour prior to transplantation.

19. The method 17, wherein the immunosuppressive agent and the anti-αβ T cell receptor antibodies are independently administered daily or non-daily for about 100 days after transplantation.

20. The method of claim 17, wherein the immunosuppressive agent and the anti-αβ T cell receptor antibodies are independently administered daily or non-daily for about 50 days after transplantation.

21. The method of claim 17, wherein the immunosuppressive agent and the anti-αβ T cell receptor antibodies are independently administered daily or non-daily for about 35 days after transplantation.

22. The method of claim 17, wherein the immunosuppressive agent and the anti-αβ T cell receptor antibodies are independently administered daily or non-daily for about 21 days after transplantation.

23. The method of claim 17, wherein the immunosuppressive agent and the anti-αβ T cell receptor antibodies are independently administered daily or non-daily for about 14 days after transplantation.

24. The method of claim 17, wherein the immunosuppressive agent and the anti-αβ T cell receptor antibodies are independently administered daily or non-daily for about 7 days after transplantation.

25. The method of claim 17, wherein the immunosuppressive agent and the anti-αβ T cell receptor antibodies are independently administered daily or non-daily for about 5 days after transplantation.

26. The method of claim 1 wherein the immunosuppressive agent and the anti-αβ T cell receptor antibodies are initially administered at about one hour prior to transplantation to about at the time of transplantation.

27. The method of claim 1 wherein the immunosuppressive agent and the anti-αβ T cell receptor antibodies are initially administered from about the time of transplantation to about three days after transplantation.

28. The method of claim 1, wherein administration of the immunosuppressive agent and the anti-αβ T cell receptor antibodies results in induction of hematopoietic mixed donor-recipient chimerism in the recipient.

29. The method of claim 1, wherein the donor is a mammal of a first species and the recipient is a mammal of a second species.

30. The method of claim 1, wherein the donor and the recipient are mammals of the same species.

31. The method of claim 1, wherein the recipient is a primate.

32. The method of claim 1, wherein the recipient is a human.

33. A method for inducing donor-specific tolerance in a semi-allogeneic transplant recipient, comprising:
(a) initially administering to a semi-allogeneic transplant recipient a therapeutically effective amount of cyclosporine A at about the time of transplantation to about 24 hours prior to transplantation;
(b) initially administering to the transplant recipient a therapeutically effective amount of anti-αβ T cell receptor antibodies about at the time of transplantation to about 24 hours prior to transplantation;
(c) implanting a semi-allogeneic allograft into the recipient; and
(d) administering a therapeutically effective amount of both cyclosporine A and anti-αβ T cell receptor antibodies independently daily or non-daily for about 5 days to about 100 days after transplantation.

34-61. (canceled)

62. A method for inducing donor-specific tolerance in a fully-allogeneic transplant recipient, comprising:
(a) initially administering to a fully-allogeneic transplant recipient a therapeutically effective amount of cyclosporine A at about the time of transplantation to about three days after transplantation;
(b) initially administering to the transplant recipient a therapeutically effective amount of anti-αβ T cell receptor antibodies at about the time of transplantation to about three days after transplantation;
(c) implanting a fully-allogeneic allograft into the recipient; and
(d) administering a therapeutically effective amount of both cyclosporine A and anti-αβ T cell receptor antibodies independently daily or non-daily for about 5 days to about 100 days after transplantation.

63-79. (canceled)
